(19) United States
(12) Patent Application Publication
(10) Pub. No.: US 2025/0195357 A1
(43) Pub. Date: Jun. 19, 2025

(54) CLEAR SCALP COMPOSITION WITH DISCRETE PARTICLES

(71) Applicant: The Procter & Gamble Company, Cincinnati, OH (US)

(72) Inventors: SUPRIYA PUNYANI, NEW DELHI (IN); JIE XIN TONG, SINGAPORE (SG); JAMES ANTHONY STAUDIGEL, LOVELAND, OH (US)

(21) Appl. No.: 18/541,516

(22) Filed: Dec. 15, 2023

Related U.S. Application Data
(60) Provisional application No. 63/433,140, filed on Dec. 16, 2022.

(51) Int. Cl. A61K 8/49; A61K 8/73; A61K 8/84; A61Q 5/00

(52) U.S. Cl. CPC A61K 8/4926; A61K 8/737; A61K 8/84; A61Q 5/006

(57) ABSTRACT

The present invention is directed to a clear scalp composition comprising from about 0.5% to about 30%, by weight, of a discrete particle comprising anhydrous particles and an aqueous phase of the clear scalp composition, wherein the anhydrous particles comprise one or more of a fatty amphiphile selected from the group consisting of fatty alcohol, fatty ester, fatty acid, fatty amide and mixtures thereof; and one or more of a surfactant selected from the group consisting of cationic, anionic, nonionic, zwitterionic or mixtures thereof; wherein the anhydrous particles comprise one or more of a scalp active; and wherein the discrete particles have a size from about 200 microns to about 25,000 microns; and an aqueous phase comprising a water soluble polymer and an aqueous carrier; wherein the clear scalp composition has a pH from about 3.5 to about 7.0.

### FIELD OF THE INVENTION

The present invention relates to discrete particles delivering scalp benefits from a clear scalp composition.

### BACKGROUND OF THE INVENTION

There is a segment of consumers who suffer from dandruff but are still reluctant to use anti-dandruff shampoos because they do not like one or more performance aspects of typical anti-dandruff shampoos (i.e. hair feel, scent, etc,). Many consumers do not want to give up their favorite cosmetic shampoo which they use for hair benefits. While today's cosmetic shampoos provide hair benefits such as hair shine, feel, and appearance, they cannot resolve flakes or dandruff. The present invention is based on the development of discrete particles containing scalp actives (anti-dandruff) in a formula which can be mixed with any consumer's shampoo product to provide scalp benefits (anti-dandruff, anti-itch).

### SUMMARY OF THE INVENTION

The present invention is directed to a clear scalp composition comprising from about 0.5% to about 30%, by weight, of a discrete particle comprising anhydrous particles and an aqueous phase of the clear scalp composition, wherein the anhydrous particles comprise one or more of a fatty amphiphile selected from the group consisting of fatty alcohol, fatty ester, fatty acid, fatty amide and mixtures thereof; and one or more of a surfactant selected from the group consisting of cationic, anionic, nonionic, zwitterionic or mixtures thereof; wherein the anhydrous particles comprise one or more of a scalp active; and wherein the discrete particles have a size from about 200 microns to about 25,000 microns; and an aqueous phase comprising a water soluble polymer and an aqueous carrier; wherein the clear scalp composition has a pH from about 3.5 to about 7.0.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Rinse-off” in reference to compositions, means compositions intended to be applied to keratinous substrate and subsequently removed by washing, rinsing or wiping within a few minutes or less from the application. These “rinse-off” compositions are to be distinguished from “leave-on” compositions, which are intended to be applied to and allowed to remain longer on the keratinous tissue.

“Soluble” means at least about 0.1 g of solute dissolves in 100 ml of solvent, at 25° C. and 1 atm of pressure.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

The term “polymer” as used herein shall include materials compositions to provide conditioning whether made by polymerization of one type of monomer or benefits. “Polymer,” as used herein, means a chemical formed from the polymerisation of two or more monomers. Polymer, as used herein shall include materials compositions to provide conditioning whether made by polymerization of one type of monomer or benefits. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise-both possibilities are suitable for the present invention. Except if stated otherwise, the term “polymer” used herein includes any type of polymer including homopolymers and copolymers.

As used herein, the term “gel network” refers to a lamellar or vesicular solid crystalline phase which comprises at least one fatty amphiphile and at least one surfactant and water or other suitable solvents, where the fatty alcohol and surfactant within this phase are arranged in multi-lamellar vesicles and/or lamellar sheets. The term “solid crystalline”, as used herein, refers to the structure of the lamellar or vesicular phase which forms at a temperature below the melt transition temperature (i.e., the chain melt temperature) of the layer in the gel network, the melt transition temperature being at least about 27° C. The melt transition temperature may be measured by differential scanning calorimetry.

The compositions and methods/processes of the present invention can comprise, consist of, and consist essentially of the essential elements and limitations of the invention described herein, as well as any of the additional or optional ingredients, components, steps, or limitations described herein.

The term “water soluble” as used herein means that the material is soluble in water in the composition. In general, the material should be soluble at 25° C. at a concentration of 0.1% by weight of the water solvent, may be at 1%, may be at 5%, and may be at 15%.

The term “discrete particles” means an anhydrous particle swollen in the presence of an aqueous phase (e.g. when the anhydrous particle is added to the cationic polymer and aqueous phase to form a scalp composition which can mixed with shampoo).

The term “anhydrous particles” as used herein means particles prepared by the co-melting and mixing of one or more surfactants and one or more fatty amphiphiles followed by cooling to solidify. The concentration of water in the anhydrous particle generally ranges from about 0% to about 30%, may be from about 0% to about 20%, may be from about 0% to about 15% by weight of the anhydrous particle. The size of the anhydrous particles in the scalp composition ranges from about 200 μm to about 25,000 μm. The scale size of the discrete particles in the scalp composition ranges from about 200 microns to about 25,000 microns, may be from about 500 μm to about 7000 μm. The scale size of the discrete particles in the scalp composition can also range from about 1000 μm to about 5000 μm. As measured by conventional techniques, visually via ruler, light microscopy etc.

The term “solid” as used herein means that the anhydrous particle at ambient temperature does not conform to the shape of the container in which the particle is held.

The term “swollen” as used herein refers to the condition of the discrete particle in the scalp composition, which particle has absorbed a sufficient quantity of aqueous solvent such that said particle either shear between fingers while pouring in-hand, on-head or mixing with other hair composition.

The term “unhydrated solid phase” as used herein refers to the portion of the discrete particle in the scalp composition that has absorbed the least amount of water and is not able to completely shear between fingers or in-hand or on-head or while mixing with other hair composition.

As used herein, “clear” composition means permitting a substantial amount of visible light to transmit through an object, for example, the clear scalp composition. Suitable light transmittance can be determined able to see through the composition without any blurriness and demonstrates <50% haze.

### Scalp Composition

The scalp composition comprises a cationic polymer, an aqueous carrier and discrete particles. The discrete particle is an anhydrous particle swollen in the presence of an aqueous phase (e.g. when the anhydrous particle is added to the detersive surfactant and aqueous phase to form a scalp booster product). The anhydrous particle comprises a fatty amphiphile, at least one surfactant, an active delivering scalp benefit (anti-dandruff, soothing, oil control, anti-itch etc.) and a low level of an aqueous phase. The low level of aqueous phase used during the formation of the anhydrous particles is from about 0% by weight of the anhydrous particle to about 30% by weight of the anhydrous particle. The scalp composition may be substantially clear and transparent.

### Discrete Particles

The scalp compositions comprise discrete particles that comprise anhydrous particles and an aqueous phase. The anhydrous particles are prepared by co-melting one or more fatty amphiphile and one or more surfactants and then cooled to solidify. Various methodologies can be used to control the particle size of such anhydrous particles. The anhydrous particles are then added into an aqueous phase that contains cationic polymer. This results in the swelling of such particles transforming them into discrete particles. This swelling can range from approximately 1 times to 100 times the original discrete particle size. In the present invention, the discrete particle may swell from about 1.5 times to about 4 times its original size when combined with the clear scalp composition. In the present invention, the discrete particle may swell to 4 times its original size when combined with the clear scalp composition.

The discrete particle can be a gel network. The swelling of discrete particle can be completed in sequence, by first soaking of discrete particle separately in aqueous solution for 0.5-3 hrs. and then add to scalp composition or simultaneously adding discrete particles into scalp composition to completely swell in 3-4 days.

The size of the discrete particles in the cleansing composition ranges from about 200 μm to about 15,000 μm. The scale size of the discrete particles in the cleansing composition ranges may be from about 500 μm to about 7000 μm. The scale size of the discrete particles in the cleansing composition ranges may be from about 1000 μm to about 5000 μm. Particle size may be measured by conventional techniques, including measuring by a caliper, visually via ruler and simple light microscopy.

Gel network beads are created by cooling bead composition in a silicone mold (silikomart Mini Pearl; item code 36.203.87.0065), resulting in gel network particles that are ˜6.4 mm in diameter. Cooled gel network beads are added to either water or the booster composition and allowed to swell for some period of time, from hours to days. This composition may contain anionic, non-ionic, amphoteric, or zwitterionic surfactant, cationic polymer, or other agents.

In the present invention, the gel network beads are added to syringes for ease of dispensing into cosmetic shampoo prior to mixing in hand. Depending on composition and time, beads can swell to the following sizes:

| Syringe | Chamber |
|---|---|
| Volume | Diameter |
| (ml) | (mm) |
| 3 | 8.9 |
| 5 | 11.4 |
| 10 | 15.2 |
| 20 | 17.8 |
| 50 | 25.4 |

### 1. Fatty Amphiphile

The anhydrous particles comprise at least one fatty amphiphile. As used herein, “fatty amphiphile” refers to a compound having a hydrophobic tail group and a hydrophilic head group which does not make the compound water soluble, wherein the compound also has a net neutral charge at the pH of the cleansing composition.

The fatty amphiphile may be characterized as a compound having a Hydrophilic-Lipophilic Balance (“HLB”) of 6 or less. The HLB, as used herein, is the standard HLB according to Griffin, J. Soc. Cosm. Chem., vol. 5, 249 (1954).

Suitable fatty amphiphiles, or suitable mixtures of two or more fatty amphiphiles, have a melting point of at least about 27° C. The melting point, as used herein, may be measured by a standard melting point method as described in U.S. Pharmacopeia, USP-NF General Chapter <741>“Melting range or temperature”. The melting point of a mixture of two or more materials is determined by mixing the two or more materials at a temperature above the respective melt points and then allowing the mixture to cool. If the resulting composite is a homogeneous solid below about 27° C., then the mixture has a suitable melting point. A mixture of two or more fatty amphiphiles, wherein the mixture comprises at least one fatty amphiphile having an individual melting point of less than about 27° C., still is suitable for use provided that the composite melting point of the mixture is at least about 27° C.

Suitable fatty amphiphiles have a hydrophobic tail group. This hydrophobic tail group may be an alkyl, alkenyl (containing up to 3 double bonds), alkyl aromatic, or branched alkyl group with a length of from about 12 to about 70 carbon atoms, and from about 16 to about 60 carbon atoms, and from about 16 to about 50 carbon atoms, and from about 16 to about 40 carbon atoms, and from about 16 to about 22 carbon atoms, and from about 18 to 22 carbon atoms. Non-limiting examples of alkyl, alkenyl, or branched alkyl groups suitable for the fatty amphiphiles include lauryl, tridecyl, myristyl, pentadecyl, cetyl, heptadecyl, stearyl, arachidyl, behenyl, undecylenyl, palmitoleyl, oleyl, palmoleyl, linoleyl, linolenyl, arahchidonyl, elaidyl, elaeostearyl, erucyl, isolauryl, isotridecyl, isomyristal, isopentadecyl, petroselinyl, isocetyl, isoheptadecyl, isostearyl, isoarachidyl, isobebnyl, gadoleyl, brassidyl, and technical-grade mixture thereof.

Suitable fatty amphiphiles also have a hydrophilic head group which does not make the compound water soluble, such as in compounds having an HLB of 6 or less. Non-limiting examples of classes of compounds having such a hydrophilic head group include fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxyalted amines, fatty carbamates, fatty amine oxides, fatty acids, alkoxylated fatty acids, fatty diesters, fatty sorbitan esters, fatty sugar esters, methyl glucoside esters, fatty glycol esters, mono, di & tri glycerides, polyglycerine fatty esters, alkyl glyceryl ethers, propylene glycol fatty acid esters, cholesterol, ceramides, fatty silicone waxes, fatty glucose amides, fatty phosphate esters, and phospholipids. For additional discussion of fatty amphiphiles which are suitable for use, see U.S. 2006/0024256 A1.

To form the anhydrous particles individual fatty amphiphile compounds or combinations of two or more different fatty amphiphile compounds may be selected.

The discrete particle is added into an aqueous solution of cationic polymer to result in a scalp composition. Therefore, the scalp compositions can comprise fatty amphiphile in an amount from about 0.05% to about 20%, may be from about 0.5% to about 10%, and may be from about 1% to about 8%, by weight of the scalp composition.

The discrete particle, when hydrated, can form a gel network in the scalp composition. The weight ratio of the fatty amphiphile to the surfactant in the gel network component is greater than about 1:9, may be greater than about 1:5 to about 100:1, may be greater than about 1:2 to about 50:1, and may be greater than about 1:1 to about 10:1.

In the present invention, with regard to making of a product such as a scalp composition, a swellable particle of the present invention may enable packing of product such as scalp composition on normal packing equipment vs. swelling larger particle, and then trying to be able to get such a product into a package due to size and increased fragility of swollen bead.

### 2. Surfactant Used for the Preparation of the Anhydrous Particles

The anhydrous particles can also comprise one or more surfactants. This surfactant which is combined with the fatty amphiphile to form the anhydrous particle are co-melted and mixed and then cooled to produce the anhydrous particles.

The scalp compositions comprise surfactant as part of the anhydrous particles in an amount from about 0.01% to about 50%, may be from about 0.1% to about 10%, and may be from about 0.3% to about 5%, by weight of the scalp composition.

Suitable surfactants include cationic, anionic, zwitterionic, amphoteric, and nonionic surfactants. The surfactant is selected from cationic, anionic, and nonionic surfactants, and mixtures thereof. For additional discussion of surfactants which are suitable for use, see U.S. 2006/0024256 A1.

Additionally, certain surfactants which have a hydrophobic tail group with a chain length of from about 16 to about 22 carbon atoms may be selected to contribute to obtaining a melt transition temperature of at least about 38° C. for the resulting anhydrous particles. For such surfactants, the hydrophobic tail group may be alkyl, alkenyl (containing up to 3 double bonds), alkyl aromatic, or branched alkyl.

Mixtures of more than one surfactant of the above specified types may be used for the surfactant.

The discrete particle may also comprise a surfactant. As used herein, “surfactant” refers to one or more surfactants which are combined with the fatty amphiphile and water for a discrete particle.

### 3. Water or Suitable Solvents

The anhydrous particle may further comprise from about 0 wt % to about 30 wt %, may be from about 1 wt % to about 20 wt %, may be from about 0 wt % to about 5 wt %, may be from about 1 wt % to about 10 wt % of water or a suitable solvent, by weight of the anhydrous particle. As used herein, the term “suitable solvent” refers to any solvent which can be used in the place of or in combination with water.

### Scalp Composition Base

The discrete particle may be added into a scalp composition base. The scalp base can comprise a cationic polymer, monocarboxylic acid, an aqueous carrier and additional components such as scalp actives.

The anhydrous particles when added to the scalp composition base (which comprise an aqueous phase) become discrete particles. The discrete particles then swell in the aqueous phase. The aqueous phase comprises water and/or suitable solvents. As used herein, the term “suitable solvent” refers to any solvent which can be used in the place of or in combination with water.

In the present invention, the discrete particles may further be in the form of a single unit which may be used as a stand-alone product or combined with a personal care product, such as a shampoo, in a person's hand prior to applying to hair and scalp.

### Cationic Polymer

The scalp composition may include a cationic polymer. The concentration of the cationic polymer in the scalp composition can be from about 0.01% to about 5%, may be from about 0.075% to about 2.5%, may be from about 0.1% to about 1.0%, and may be from about 0.3% to about 1.0% by weight of the cleansing composition.

Suitable cationic polymers may have cationic charge densities of at least about 0.4 meq/g, may be at least about 0.7 meq/g, may be at least about 1.2 meq/g, may be at least about 1.5 meq/g, may be less than about 7 meq/g, and may be less than about 5 meq/g, at the pH of intended use of the composition. The pH will generally range from about pH 3 to about pH 9, may be between about pH 4 and about pH 8. The “cationic charge density” of a polymer, as that term is used herein, refers to the ratio of the number of positive charges on the polymer to the molecular weight of the polymer. The weight average molecular weight of such suitable cationic polymers will generally be between about 10,000 and 10 million, may be between about 50,000 and about 5 million, and may be between about 100,000 and about 3 million.

Suitable cationic polymers for use in the composition include polysaccharide polymers, such as cationic cellulose derivatives and cationic starch derivatives, such as salts of hydroxyethyl cellulose reacted with trimethyl ammonium substituted epoxide. Other suitable cationic polymers include cationic guar gum derivatives, such as guar hydroxypropyltrimonium chloride. Further suitable cationic polymers include galactomannan polymer derivatives having a mannose to galactose ratio of greater than 2:1 on a monomer to monomer basis, such as cassia gum hydroxypropyltrimonium chloride. Particularly suitable cationic deposition polymers include guar hydroxypropyltrimonium chloride.

The cationic guar polymer may be formed from quaternary ammonium compounds. The quaternary ammonium compounds for forming the cationic guar polymer conform to the general formula 1:

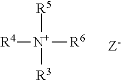

wherein where R3, R4 and R5 are methyl or ethyl groups; R6 is either an epoxyalkyl group of the general formula 2:

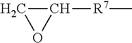

or R6 is a halohydrin group of the general formula 3:

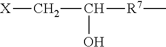

wherein R7 is a C1 to C3 alkylene; X is chlorine or bromine, and Z is an anion such as Cl—, Br—, I— or HSO4-.

The cationic guar polymer can conform to the general formula 4:

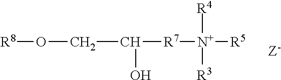

wherein R8 is guar gum; and wherein R4, R5, R6 and R7 are as defined above; and wherein Z is a halogen. The cationic guar polymer can conform to Formula 5:

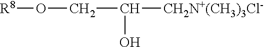

Suitable cationic guar polymers include cationic guar gum derivatives, such as guar hydroxypropyltrimonium chloride. The cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-17, which has a cationic charge density of about 0.6 meq/g and a M. Wt. of about 2.2 million g/mol and is available from Rhodia Company. Jaguar® C 13S which has a M. Wt. of 2.2 million g/mol and a cationic charge density of about 0.8 meq/g (available from Rhodia Company). N-Hance 3196, which has a charge density of about 0.7 and a M. Wt. Of about 1,100,000 g/mole and is available from ASI. BF-13, which is a borate (boron) free guar of charge density of about 1.1 meq/g and M. W.t of about 800,000 and BF-17, which is a borate (boron) free guar of charge density of about 1.7 meq/g and M. W.t of about 800,000 both available from ASI.

A combination of cationic polymers can improve the conditioning of the scalp composition and act as suspending agent for discrete particles. Using a cationic polymer with a charge density of from about 0.4 meq/g to about 0.8 meq/g, may be about 0.7 meq/g in combination with a cationic polymer having a molecular weight greater than about 1,000,000 can result in a scalp composition with clean wet hair feel.

The scalp composition can comprise polyquaternium-37, i.e., homopolymer of N,N,N-trimethyl-2-((2-methyl-1-oxo-2-propenyl)oxy)-chloride homopolymer.

The polyquaternium-37 useful herein has a cationic charge density of, from about 2.3 meq/g, may be from about 4.5 meq/g, may be from about 5.8 meq/g, and may be to about 13 meq/g, may be to about 10 meq/g, may be to about 7.0 meq/g.

The polyquaternium-37 useful herein has a molecular weight of, from about 207.7 g/mol or more, may be from about 250 g/mol or more, may be from about 200 g/mol or more in view of providing improved hair conditioning, anti-static.

Commercially available examples of polyquaternium-37 polymer include, for a non-limiting example, that having a tradename COSMEDIA® Ultragel 300 available from BASF.

The scalp composition may comprise diallyldimethylammonium chloride (DADMAC).

### Aqueous Carrier

The scalp composition may comprise an aqueous carrier. Typically, the composition is in the form of pourable liquids (under ambient conditions). The composition, therefore, comprises an aqueous carrier at a level of at least about, may be from about 20% to about 95%, and may be from about 60% to about 85%, by weight of the compositions. The aqueous carrier may comprise water, or a miscible mixture of water and organic solvent. The aqueous carrier may also comprise water with minimal or no significant concentrations of organic solvent, except as otherwise incidentally incorporated into the composition as minor ingredients of other components.

### Detersive Surfactant

The scalp composition of the present invention may comprise greater than about 10% by weight of a surfactant system which provides cleaning performance to the composition, and may be greater than 12% by weight of a surfactant system which provides cleaning performance to the composition. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 25%, from about 10% to about 18%, from about 10% to about 14%, from about 10% to about 12%, from about 11% to about 20%, from about 12% to about 20%, and/or from about 12% to about 18% by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the personal care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:
a) R₁ O (CH₂CHR₃O)_{y}SO₃M;
b) CH₃ (CH₂)_{z} CHR₂ CH₂ O (CH₂ CHR₃O)_{y}SO₃M; and
c) mixtures thereof,
where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

The scalp composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The scalp composition may further comprise from about 0.25% to about 15%, from about 1% to about 14%, from about 2% to about 13% by weight of one or more amphoteric, zwitterionic, nonionic co-surfactants, or a mixture thereof.

Suitable amphoteric or zwitterionic surfactants for use in the personal care composition herein include those which are known for use in shampoo or other personal care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, those selected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The composition may comprise a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Texas under the trade names Neodol® 91, Neodol® 23, Neodol® 25, Neodol® 45, Neodol® 135, Neodol® 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Delaware, including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tx.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tx.) and octylphenol ethoxylates (Triton™ X-15, X-35, X-45, X-114, X-100, X-102, X-165, X-305, X-405, X-705 available from The Dow Chemical Company, Houston, TX).

Also suitable for use herein are tertiary alkylamine oxides including lauramine oxide and cocamine oxide.

Non limiting examples of other anionic, zwitterionic, amphoteric, and non-ionic additional surfactants suitable for use in the personal care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, alternatively from about 1% to about 25%, alternatively from about 2% to about 20%. The surfactant combination can have a cumulative average weight % of C8 to C12 alkyl chain lengths of from about 7.5% to about 25%, alternatively from about 10% to about 22.5%, alternatively from about 10% to about 20%. The surfactant combination can have an average C8-C12/C13-C18 alkyl chain ratio from about 3 to about 200, alternatively from about 25 to about 175.5, alternatively from about 50 to about 150, alternatively from about 75 to about 125.

### Additional Components

The scalp composition may further comprise one or more optional components known for use in hair care or personal care products, provided that the optional components are physically and chemically compatible with the components described herein, or do not otherwise unduly impair product stability, aesthetics or performance. Individual concentrations of such optional components may range from about 0.001% to about 10% by weight of the compositions.

Non-limiting examples of optional components for use in the composition include cationic polymers, conditioning agents (hydrocarbon oils, fatty esters, silicones), anti-dandruff agents, sensates, suspending agents, viscosity modifiers, dyes, nonvolatile solvents or diluents (water soluble and insoluble), pearlescent aids, foam boosters, additional surfactants or nonionic co-surfactants, pediculocides, pH adjusting agents, perfumes, preservatives, chelants, proteins, skin active agents, sunscreens, UV absorbers, and vitamins.

### A. Dispersed Particles

The composition may include dispersed particles. Particles useful can be inorganic, synthetic, or semi-synthetic in origin. If present, dispersed particles are incorporated in an amount from about 0.025% to about 20%, may be from about 0.05% to about 10%, may be from about 0.1% to about 5%, may be from about 0.25% to about 3%, and may be from about 0.5% to about 1%, by weight of the composition.

### B. Cationic Surfactant

The scalp composition of the present invention may comprise a cationic surfactant. In the present invention, the cationic surfactant may be Behentrimonium Methosulfate, BTMS.

The cationic surfactant can be one cationic surfactant or a mixture of two or more cationic surfactants. The cationic surfactant system may be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt. The cationic surfactant system may be a mixture of mono-long alkyl quaternized ammonium salt and di-long alkyl quaternized ammonium salt.

### Mono-Long Alkyl Quaternized Ammonium Salt

The monoalkyl quaternized ammonium salt cationic surfactants useful herein are those having one long alkyl chain which has from 12 to 30 carbon atoms, may be from 16 to 24 carbon atoms, and may be from C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

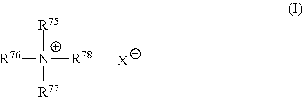

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ may be selected from an alkyl group of from 12 to 30 carbon atoms, may be from 16 to 24 carbon atoms, may be from 18 to 22 carbon atoms, may be from 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of the acid discussed, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with mono carboxylic acid such as l-glutamic acid, salicylic acid, lactic acid, hydrochloric acid, malic acid, succinic acid, acetic acid, fumaric acid, tartaric acid, glycolic acid, aspartic acid, citric acid, l-glutamic hydrochloride, maleic acid, lactobionic acid or glucolactone and mixtures thereof; may be salicylic acid, l-glutamic acid, lactic acid, aspartic acid, glycolic acid, lactobionic acid or glucolactone. The amines herein may be partially neutralized with any of the acids at a molar ratio of the amine to the acid of from about 1:1 to about 1:4, may be from about 1:1.1 to about 1:3.2.

### Di-Long Alkyl Quaternized Ammonium Salt

Di-long alkyl quaternized ammonium salt may be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range may be from about 10% to about 50%, may be from about 30% to about 45%.

The dialkyl quaternized ammonium salt cationic surfactants useful herein are those having two long alkyl chains having 12-30 carbon atoms, may be 16-24 carbon atoms, may be 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

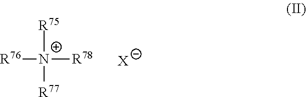

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ may be selected from an alkyl group of from 12 to 30 carbon atoms, may be from 16 to 24 carbon atoms, may be from 18 to 22 carbon atoms, may be from 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### C. Scalp Active

Piroctone and salts of piroctone, such as piroctone olamine, are known to provide anti-dandruff benefit and is a scalp active. However, piroctone olamine is often supplied as a crystal powder, and is also known to have very limited solubility in water, meaning the challenge is to assure solubilization of an anti-dandruff active in as simple as possible a material combination.

It has been found by the present invention that, certain solid organic compounds, such as piroctone and/or salts thereof, tend to crystallize in the following conditions:
when the aqueous composition comprises cationic surfactant and high melting point fatty compounds;
when the amount of the solid organic compounds increases in the composition, especially 0.2% or above; and/or
when the composition has pH of from about 3.5 to about 7.

Piroctone olamine in a conditioner is usually solubilized by some type of oil at high concentration (around 5%). The oil is typically emulsified by a cationic surfactant and high melting point fatty compounds, resulting in a highly viscous and opaque product. In contrast, the present invention does not use such oil (which causes the hair and scalp to become oily), and instead uses a specific surfactant body, such as BTMS, and a specific acid as the counterion, in a specific ratio of the surfactant body/acid/piroctone olamine, to get to a desired pH range for the piroctone olamine to function and deposit better on the scalp. This leads to anti-dandruff efficacy and good conditioning, while not using too much of the cationic surfactant and high melting point fatty compounds.

In the present invention, the scalp active may be a substituted 2-pyridinol-N-oxide material, such as piroctone olamine or ciclopirox olamine. Piroctone olamine and ciclopirox olamine are the salt of piroctone and ciclopirox, respectively, with monoethanolamine. In the compositions having an acidic pH, they become protonated to become piroctone and ciclopirox, respectively. In the present invention, the composition may have an acidic pH, may have a pH from about 2 to about 7, may be from about 3.5 to about 7, may be from about 3.5 to about 6, furthermore may be from about 3.5 to about 5.

The present invention may include one or more scalp actives, which may be one material or a mixture selected from the groups consisting of: azoles, such as climbazole, ketoconazole, itraconazole, econazole, and elubiol; hydroxy pyridones, such as piroctone olamine, ciclopirox, rilopirox, and MEA-Hydroxyoctyloxypyridinone; strobilurins such as azoxystrobin and metal chelators such as 1,10-phenanthroline, salicylic acid and menthol.

The azole soluble scalp active may be an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. The azole soluble scalp health active may be ketoconazole. The sole soluble scalp health active may be ketoconazole.

These scalp active compounds are not soluble in a typical solvent such as water or an aqueous mixture such as water containing even a high level of glycerin or propylene glycol. They typically require either a high level (greater than 5% or even greater than 10%) of an emollient oil, such as triethylhexanoine, octyldodecyl myristate, or a high level (greater than 50%) of an alcohol, such as ethanol. The former (oil) is known to cause an oily feel that is a negative on the scalp and hair, and for the latter (alcohol), it is known that most consumers are afraid to apply to a scalp that is already sensitive due to dandruff symptoms. The present invention solubilizes such scalp actives by the combination of a surfactant (primarily needed for hair conditioning), and a specific acid (primarily needed to adjust pH). Nothing other than these two materials are required to formulate these anti-dandruff and scalp actives into soluble form in a liquid.

In the present invention, the scalp active may be a particulate in a fatty acid matrix. Pyridinethione particulates are suitable particulate scalp actives for use in composition of the present invention. In the present invention, the anti-dandruff active may be a 1-hydroxy-2-pyridinethione salt and is in particulate form. In the present invention, the concentration of pyridinethione particulate may range from about 0.01% to about 5%, by weight of the composition, or from about 0.1% to about 3%, or from about 0.1% to about 2%. In the present invention, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”; zinc pyrithione).

In the present invention, salicylic acid may be water soluble at pH of 4.8 and above. In the present invention, piroctone olamine and menthol are oil soluble.

In the present invention, the scalp active could also be sulfur or selenium sulfide. In addition salicylic acid may be combined with piroctone olamine, zinc pyrithione, sulfur, selenium sulfide or any combinations thereof.

Any of the scalp actives may be in the composition in an amount from about 0.01% to about 3%, by weight of the composition; may be from about 0.1% to 2%, or may be from about 0.25% to about 1%, or may be from about 0.5% to 1% of antidandruff or other scalp actives.

### D. Rheology Modifier

In the present invention, the scalp composition may comprise a rheology modifier to increase the substantivity and stability of the composition. Any suitable rheology modifier can be used. In the present invention, the scalp composition may comprise from about 0.05% to about 10% of a rheology modifier, or from about 0.1% to about 10% of a rheology modifier, or from about 0.5% to about 2% of a rheology modifier, or from about 0.7% to about 2% of a rheology modifier, and/or from about 1% to about 1.5% of a rheology modifier. In the present invention, the rheology modifier may be an ethoxylated (20) sorbitan ester based such as tween 20. In the present invention, the rheology modifier may be a polymeric rheology modifier.

In the present invention, the scalp composition may comprise rheology modifiers that are homopolymers based on acrylic acid, methacrylic acid or other related derivatives, non-limiting examples include polyacrylate, polymethacrylate, polyethylacrylate, and polyacrylamide.

In present invention, the rheology modifiers may be alkali swellable and hydrophobically-modified alkali swellable acrylic copolymers or methacrylate copolymers non-limiting examples include acrylic acid/acrylonitrogen copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/vinylneodecanoate crosspolymer, and acrylates/C10-C30 alkyl acrylate crosspolymer.

In the present invention, the rheology modifiers may be crosslinked acrylic polymers, a non-limiting example includes carbomers.

In the present invention, the rheology modifiers may be alginic acid-based materials; non-limiting examples include sodium alginate, and alginic acid propylene glycol esters.

In the present invention, the rheology modifier may be an associative polymeric thickeners, non-limiting examples include: Hydrophobically modified cellulose derivatives; Hydrophobically modified alkoxylated urethane polymers, nonlimiting example include PEG-150/decyl alcohol/SMDI copolymer, PEG-150/stearyl alcohol/SMDI copolymer, polyurethane-39; Hydrophobically modified, alkali swellable emulsions, non-limiting examples include hydrophobically modified polyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from 10-300, or from 30-200, and/or from 40-150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

In the present invention, the rheology modifier may be cellulose and derivatives; nonlimiting examples include microcrystalline cellulose, carboxymethylcelluloses, hydroxyethylcellulose, hydroxypropylcellulose, hydroxypropylmethylcellulose, methylcellulose, ethylcellulose, nitro cellulose, cellulose sulfate, cellulose powder, and hydrophobically modified celluloses.

In the present invention, the rheology modifier may be a guar and guar derivatives; nonlimiting examples include hydroxypropyl guar, and hydroxypropyl guar hydroxypropyl trimonium chloride.

In the present invention, the rheology modifier may be polyethylene oxide, polypropylene oxide, and POE-PPO copolymers.

In the present invention, the rheology modifier may be polyvinylpyrrolidone, crosslinked polyvinylpyrrolidone and derivatives. In the present invention, the rheology modifier may be polyvinyalcohol and derivatives.

In the present invention, the rheology modifier may be polyethyleneimine and derivatives.

In the present invention, the rheology modifier may be silicas; nonlimiting examples include fumed silica, precipitated silica, and silicone-surface treated silica.

In the present invention, the rheology modifier may be water-swellable clays non-limiting examples include laponite, bentolite, montmorilonite, smectite, and hectonite.

In the present invention, the rheology modifier may be gums nonlimiting examples include xanthan gum, guar gum, hydroxypropyl guar gum, Arabia gum, tragacanth, galactan, carob gum, karaya gum, and locust bean gum.

In the present invention, the rheology modifier may be, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of rheology modifiers include acrylamide/ammonium acrylate copolymer (and) polyisobutene (and) polysorbate 20, acrylamide/sodium acryloyldimethyl taurate copolymer/isohexadecane/polysorbate 80, acrylates copolymer; acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil, C12-16 alkyl PEG-2 hydroxypropylhydroxyethyl ethylcellulose (HM-EHEC), carbomer, crosslinked polyvinylpyrrolidone (PVP), dibenzylidene sorbitol, hydroxyethyl ethylcellulose (EHEC), hydroxypropyl methylcellulose (HPMC), hydroxypropyl (HPMC), methylcellulose hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7; polyacrylate 13/polyisobutene/polysorbate 20; polyacrylate crosspolymer-6, polyamide-3; polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol Ulterez 30, Carbopol 1342, Sepigel™ 305, Simulgel™600, Sepimax Zen, and combinations thereof.

### E. Additional Conditioning Agents

The compositions may also comprise one or more conditioning agents which are in addition to the conditioning delivered by the discrete particles. Conditioning agents include materials which are used to give a particular conditioning benefit to hair and/or skin. The conditioning agents useful in the compositions typically comprise a water-insoluble, water-dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix.

The scalp composition may further comprise a non-volatile silicone oil. For an opaque composition of the present invention, the cleansing composition comprises a non-volatile silicone oil having a particle size as measured in the cleansing composition from about 1 μm to about 50 μm. The cleansing composition may also comprise a non-volatile silicone oil having a particle size as measured in the cleansing composition from about 100 nm to about 1 μm. For a substantially clear composition of the present invention, the scalp composition may comprise a non-volatile silicone oil having a particle size as measured in the scalp composition of less than about 100 nm.

When present, the one or more conditioning agents are in an amount from about 0.01% to about 10%, may be from about 0.1% to about 8%, and may be from about 0.2% to about 4%, by weight of the composition.

The conditioning agents may be present in the discrete particle, or may be added to the final scalp composition as a separate component such that they are present primarily in the continuous phase of the composition.

### F. High Melting Point Fatty Compound

The composition of the present invention may comprise a high melting point fatty compound. The high melting point fatty compound is included in the composition at a level of which may be from about 0.1% to about 20%, may be from about 1% to about 15%, still more may be from about 1.5% to about 8% by weight of the composition,

The high melting point fatty compound useful herein have a melting point of 25° C. or higher to 60° C., may have a melting point of 30° C. to 60° C., or may have a melting point of 40° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols may be used in the composition of the present invention. The fatty alcohols useful herein are those having from about 14 to about 30 carbon atoms, may be from about 16 to about 22 carbon atoms. These fatty alcohols are saturated and can be straight or branched chain alcohols. The present invention may include fatty alcohols, for example, cetyl alcohol, stearyl alcohol, behenyl alcohol, and mixtures thereof.

High melting point fatty compounds of a single compound of high purity may be used in the present invention. Single compounds of pure fatty alcohols selected from the group of pure cetyl alcohol, stearyl alcohol, and behenyl alcohol may be used in the present invention. By “pure” herein, what is meant is that the compound has a purity of at least about 90%, may be from at least about 95%. These single compounds of high purity provide good rinsability when the consumer rinses off the composition.

### Other Optional Components

The compositions may contain other optional components. Optional components may be present in the dispersed gel network phase or may be added to the final cleansing composition as separate components.

For example, the compositions may contain water-soluble and water-insoluble vitamins such as vitamins B1, B2, B6, B12, C, pantothenic acid, pantothenyl ethyl ether, panthenol, biotin and their derivatives, and vitamins A, D, E, and their derivatives. The compositions may also contain water-soluble and water-insoluble amino acids such as asparagine, alanine, indole, glutamic acid and their salts, and tyrosine, tryptamine, lysine, histadine and their salts. The compositions may further comprise materials useful for hair loss prevention and hair growth stimulants or agents.

Any other suitable optional component can also be included in the composition, such as those ingredients that are conventionally used in given product types. The CTFA Cosmetic Ingredient Handbook, Tenth Edition (2004), published by the Cosmetic, Toiletry, and Fragrance Association, Inc., Washington, D.C., describes a wide variety of nonlimiting materials that can be added to the composition herein. Examples of these ingredient classes include, but are not limited to: abrasives, absorbents, aesthetic components such as perfumes and fragrances, pigments, colorings/colorants, essential oils, skin sensates, astringents, etc. (e.g., clove oil, menthol, camphor, Eucalyptus oil, eugenol, menthyl lactate, witch hazel distillate), anti-acne agents, anti-caking agents, antifoaming agents, antimicrobial agents (e.g., iodopropyl butylcarbamate), antibacterial agents, antifungal agents, antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, plant derivatives, plant extracts, plant tissue extracts, plant seed extracts, plant oils, botanicals, botanical extracts, preservatives, propellants, reducing agents, sebum control agents, sequestrants, skin bleaching and lightening agents, (e.g., hydroquinone, kojic acid, ascorbic acid, magnesium ascorbyl phosphate, ascorbyl glucoside, pyridoxine), enzymes, coenzymes, skin-conditioning agents (e.g., humectants and occlusive agents), skin soothing and/or healing agents and derivatives (e.g., panthenol, and derivatives such as ethyl panthenol, aloe vera, pantothenic acid and its derivatives, allantoin, bisabolol, and dipotassium glycyrrhizinate), skin treating agents (e.g., vitamin D compounds, mono-, di-, and tri-terpenoids, beta-ionol, cedrol), thickeners (including a mono- or divalent salt such as sodium chloride), and vitamins, their derivatives, and combinations thereof.

### Formulations and Examples

The following are non-limiting examples of the present invention. The examples are given solely for the purpose of illustration and are not to be construed as limitations of the present invention, as many variations thereof are possible without departing from the spirit and scope of the invention, which would be recognized by one of ordinary skill in the art.

### Formulation Preparation and Treatment Protocols to Form Discrete Particles Containing Scalp Active

### Discrete Particles Preparation:

The discrete particle composition is prepared by adding the scalp active (piroctone olamine) in an oil soluble matrix (cationic surfactant with fatty alcohol or anionic surfactant), wherein all other materials (rheology modifier, pigment, sensate) are also added in a vessel and heated to 80° C. to melt them into a homogenous mixture. Once the melt is a homogenous solution it is poured into a mold to form a particular shape or spread onto a sheet, ground into small shapes, and sieved to achieve the desired size. Discrete particle can be made of SLS (sodium lauryl sulfate) powder, ceterateh-25 and fatty alcohol (cetyl and stearyl alcohol) in 1:1:1.

### Scalp Composition Preparation:

The scalp composition is able to suspend the discrete particles and delivers consumer desired shampooing upon mixing with other shampoo and scalp benefit (anti-dandruff).

The scalp composition comprises from about 0.01% to about 5%, may be from about 0.05% to about 3%, may be from about 0.1% to about 2% of a compound selected from the group consisting of polyquaternium polymers or anionic surfactant and mixtures thereof, by weight of the scalp composition. After mixing this to other hair care product composition and applying on scalp and hair, the method then comprises rinsing the scalp composition from the hair.

The swelling of discrete particle can be done in sequence, by first soaking of discrete particle separately in aqueous solution for 0.5-3 hrs. and then add to scalp composition or simultaneously adding discrete particles into scalp composition to completely swell in 3-4 days.

### Evaluation Methods

The evaluation of discrete particle composition in a scalp composition using the following methodology.

Discrete Particle stability: Sample is stored in glass jar at room temperature. Visible change in particle shape/size/appearance is recorded everyday and after 1 month. S=Stable, U=Unstable.

Discrete Particle Spreadability: Spreadability of particle is scored within a day of sample making. Spreadability score is scored by panel for the formula, 0-hard to spread and 5-very easy/soft to spread in-hand using fingers.

### Compression Measurement of Discrete Particles

A method described herein, characterizes the degree to which the discrete particles have softened in product. This method employs the Kawabata KES-FB3-Auto Compression Tester, which is manufactured by Kato Tech, LTD, or like equipment. Following, are the equipment settings employed herein for the Kawabata KES-FB3-Auto Compression Tester-Sensitivity: 2, Velocity: 0.02 mm/sec, Stroke 10, Zone or Probe Compression Area: 2 (corresponds to a standard 16 mm compression probe), Process Rate: 0.1, Maximum Load: 50 gf/cm2.

### Sample Preparation for Compression Measurement

Product samples are prepared for measurement by incorporating discrete particles into finished product (e.g. at a ratio of about 10 discrete particles into about 5 grams of cleansing composition such as a shampoo) such that the discrete particles are effectively coated by product. These discrete particles are sampled from product one at a time at various time intervals using a small spatula, taking care not to deform the discrete particles during extraction. Without being limited to a specific time scale, discrete particle compression is performed at the following time intervals (hrs.): 0, 1, 3, 6 and 24 hours. Following proper equipment calibration and measurement set-up, a discrete particle is carefully extracted from product and centered on the sample stage of the KES-FB3-A Compression Tester, beneath the compression probe, in preparation for sample analysis.

### Formulation Examples of Discrete Particle Composition

| | Formula Example | | | | | | |
|---|---|---|---|---|---|---|---|
| | Control | | | | | | |
| | Ex 1 | Ex2 | Ex3 | Ex4 | Ex5 | Ex6 | Ex7 |
| Cetyl Alcohol¹ | 1.26 | 1.26 | 1.26 | 1.26 | 1.26 | 1.26 | 1.26 |
| Stearyl Alcohol² | 3.31 | 3.31 | 3.31 | 3.31 | 3.31 | 3.31 | 3.31 |
| Behentrimonium | 3.22 | 3.22 | 3.22 | 3.22 | 3.22 | 3.22 | 3.22 |
| Methosulfate - | | | | | | | |
| IPA³ | | | | | | | |
| Water | 0.08 | 0.08 | 0.08 | 0.08 | 0.08 | 0.08 | 0.08 |
| Pigment⁴ | 0.08 | 0.08 | 0.08 | 0.08 | 0.08 | 0.08 | 0.08 |
| Tween 20⁵ | | | | 0.1 | 0.1 | 0.1 | 0.1 |
| Piroctone Olamine⁶ | | 0.8 | 0.8 | 0.8 | 0.8 | 0.8 | 0.8 |
| Citric acid⁷ | | | | | | 0.92 | 0.92 |
| Benzyl alcohol⁸ | 0.41 | 0.41 | 0.44 | 0.44 | 0.44 | 0.44 | 0.44 |
| Discrete Size | 1.5 cm | 1.1 cm | 0.9 cm | 1.1 cm | 1.7 cm | 1.8 cm | 1.5 mm |
| (cm/mm) | | | | | | | |
| Discrete Particle | Stable | Unstable | Stable | Stable | Unstable | Stable | Stable |
| Stability | | | | | | | |
| pH of discrete | 4.5 | 8 | 5 | 5 | 5 | 5 | 5 |
| particle in scalp | | | | | | | |
| composition | | | | | | | |
| Discrete Particle | 4 | 4 | 3 | 4 | 5 | 5 | 5 |
| Spreadability (1 - | | | | | | | |
| non-speadable to 5 - | | | | | | | |
| easily spreadable) | | | | | | | |
| Discrete Particle in | 5 | 2 | 2 | 4 | 4 | 5 | 5 |
| Scalp Composition | | | | | | | |
| mixing with other | | | | | | | |
| shampoo (1-non- | | | | | | | |
| homogenous and 5- | | | | | | | |
| homogenous) | | | | | | | |

¹Supplied by P&G Chemicals
²Supplied by P&G Chemicals
³Supplied by Feixiang Chemicals (Zhangjingang) Co., Ltd.
⁴UNIPURE BLUE LC686 (Sensient)
⁵GPR RECTAPUR ®, VWR Chemicals
⁶Octopirox, Clariant
⁷Jungbunzlauer Austria
⁸Supplied by Ineos Maastricht BV (Maastricht NL)

Ex 2 shows the incorporation of scalp active into discrete particle Control Ex 1, causing destabilization of particle due to pH change. Ex 3 shows stabilization of the particle by stabilizing the pH of the matrix, resulting in a hard to spread particle. Ex 4 shows inclusion of a non-ionic surfactant which acts as a rheology modifier by softening the particle. Ex 5 results in a larger particle but results in instability due to over swelling of the particle in the scalp composition. Ex 6 is an inventive example wherein discrete particles are soft and stable vs. Ex 2-5. Ex 7 is a particle that is larger in size, is ground and sieved to select the desired particle size.

### Formulation Examples of Scalp Composition

| | Formula Example | | | | | | | | |
|---|---|---|---|---|---|---|---|---|---|
| | Control | | | | | | | | |
| | Ex 8 | Ex9 | Ex10 | Ex11 | Ex12 | Ex13 | Ex14 | Ex15 | Ex16 |
| PQ-37¹ | 0.4 | 0.4 | 0.8 | 0.8 | 0.8 | 0.8 | 0.8 | | |
| SLS² | | | | | | | | 3.08 | 3.08 |
| Cocamidopropyl | | | | | | | | 3.11 | 3.11 |
| Betaine | | | | | | | | | |
| Guar³ | | | | | | | | 0.27 | |
| HPMC⁴ | | | | | | | | 0.23 | 0.23 |
| Menthol | | | | | | | | 0.32 | |
| Benzyl Alcohol | | | | | | | | 0.36 | 0.36 |
| Water | 91.24 | 90.44 | 89.09 | 88.99 | 88.99 | 88.07 | 88.07 | 82.36 | 82.95 |
| Citric acid | | | 0.92 | 0.92 | 0.92 | 0.92 | 0.92 | 0.27 | 0.27 |

| | Discrete particle Ex from above table | | | | | | | | |
|---|---|---|---|---|---|---|---|---|---|
| | Control | | | | | | | | |
| | Ex 1 | Ex2 | Ex3 | Ex4 | Ex5 | Ex6 | Ex7 | Ex6 | Ex7 |
| Scalp | Stable | Unstable | Stable | Stable | Stable | Stable | Stable | Stable | Stable |
| composition | | | | | | | | | |
| stability | | | | | | | | | |
| pH of scalp | 4.5 | 8 | 5 | 5 | 5 | 5 | 5 | 5 | 5 |
| composition | | | | | | | | | |
| with discrete | | | | | | | | | |
| particle | | | | | | | | | |

¹Cosmedia ® Ultragel ® 300 - BASF
²Sodium Lauryl Sulfate
³Jaguar Excel (Solvay)
⁴Methocel 40-101 (Dow Chemical)

Ex 9 demonstrates the discrete particle Ex 2 added to a scalp composition resulting in a high pH composition due to addition of Piroctone Olamine thereby causing instability. Ex10 has discrete particle Ex 3 which becomes hard while stabilizing pH of scalp composition. Ex 11 contains discrete particle Ex 4 however it shows instability due to over swelling. For example, Ex 12 with discrete particle Ex 5. Formulation Examples 13 and 14 with desired pH 5, demonstrate both discrete particle and scalp composition stability. Examples 15 and 16 embody the inclusion of anionic surfactant, amphoteric surfactant, and cationic guar to enhance the cosmetic performance of the composition.

### Combinations

A. A clear scalp composition comprising a) from about 0.5% to about 30%, by weight, of a discrete particle comprising anhydrous particles and an aqueous phase of the clear scalp composition, wherein the anhydrous particles comprise one or more of a fatty amphiphile selected from the group consisting of fatty alcohol, fatty ester, fatty acid, fatty amide and mixtures thereof; and one or more of a surfactant selected from the group consisting of cationic, anionic, nonionic, zwitterionic or mixtures thereof; wherein the anhydrous particles comprise one or more of a scalp active; and wherein the discrete particles have a size from about 200 microns to about 25,000 microns; and b) an aqueous phase comprising a water soluble polymer and an aqueous carrier; wherein the clear scalp composition has a pH from about 3.5 to about 7.0.
B. A clear scalp composition according to Paragraph A, wherein the one or more of a surfactant is a cationic surfactant.
C. A clear scalp composition according to Paragraph A-B, wherein the cationic surfactant is behentrimonium methosulfate.
D. A clear scalp composition according to Paragraph A-C, wherein the discrete particle size is from about 500 μm to about 7000 μm.
E. A clear scalp composition according to Paragraph A-D, wherein the discrete particle size is from about 1000 μm to about 5000 μm.
F. A clear scalp composition according to Paragraph A-E, wherein the scalp active is selected from the group consisting of piroctone olamine, climbazole, salicylic acid, zinc pyrithione, selenium sulfide, sulfur, menthol and mixtures thereof.
G. A clear scalp composition according to Paragraph A-F, wherein the scalp active is piroctone olamine.
H. A clear scalp composition according to Paragraph A-G, wherein the discrete particle comprises from about 0.25% to 1% of piroctone olamine.
I. A clear scalp composition according to Paragraph A-H, wherein the pH is from about 3.5 to about 5.
J. A clear scalp composition according to Paragraph A-I, wherein the water soluble polymer is a cationic polymer.
K. A clear scalp composition according to Paragraph A-J, wherein the cationic polymer is polyquaternium-37.
L. A clear scalp composition according to Paragraph A-K, wherein the cationic polymer is a cationic guar polymer.
M. A clear scalp composition according to Paragraph A-L, wherein the cationic polymer is a polquaternium-10.
N. A clear scalp composition according to Paragraph A-M, wherein the cationic polymer is a diallyldimethylammonium chloride.
O. A clear scalp composition according to Paragraph A-N, wherein the water soluble polymer is present from about 0.05% to about 5%.
P. A clear scalp composition according to Paragraph A-O, wherein the water soluble polymer is present from about 0.3% to about 1%.
Q. A clear scalp composition according to Paragraph A-P, wherein the aqueous carrier is selected from the group consisting of water, or a miscible mixture of water and an organic solvent.
R. A clear scalp composition according to Paragraph A-Q, wherein the aqueous carrier is a surfactant selected from the group consisting of anionic, amphoteric, zwitterionic or non-ionic surfactants.
S. A clear scalp composition according to Paragraph A-R, wherein a swelling of the discrete particle is completed in sequence, by first soaking of the discrete particle separately in an aqueous solution for 0.5-3 hrs and then add to clear scalp composition.
T. A clear scalp composition according to Paragraph A-S, wherein a swelling of the discrete particle is completed by simultaneously adding the discrete particle into the clear scalp composition to completely swell in 3-4 days.
U. A clear scalp composition according to Paragraph A-T, wherein the clear scalp composition is clear demonstrating demonstrates <50% haze.
V. A clear scalp composition according to Paragraph A-U, wherein the discrete particles are spreadable between fingers on a hand when sheared and is stable.
W. A clear scalp composition according to Paragraph A-V, wherein the discrete particle is a swellable particle which enables packing of the clear scalp composition on packing equipment.
X. A clear scalp composition according to Paragraph A-W, wherein the discrete particle swells from about 1.5 times to about 4 times its original size when combined with the clear scalp composition.
Y. A clear scalp composition according to Paragraph A-X, wherein the discrete particle swells to 4 times its original size when combined with the clear scalp composition.

It will be appreciated that other modifications of the present disclosure are within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A clear scalp composition comprising:
a) from about 0.5% to about 30%, by weight, of a discrete particle comprising anhydrous particles and an aqueous phase of the clear scalp composition, wherein the anhydrous particles comprise one or more of a fatty amphiphile selected from the group consisting of fatty alcohol, fatty ester, fatty acid, fatty amide and mixtures thereof; and one or more of a surfactant selected from the group consisting of cationic, anionic, nonionic, zwitterionic or mixtures thereof; wherein the anhydrous particles comprise one or more of a scalp active; and wherein the discrete particles have a size from about 200 microns to about 25,000 microns; and
b) an aqueous phase comprising a water soluble polymer and an aqueous carrier;
wherein the clear scalp composition has a pH from about 3.5 to about 7.0.

2. A clear scalp composition according to claim 1 wherein the one or more of a surfactant is a cationic surfactant.

3. A clear scalp composition according to claim 2 wherein the cationic surfactant is behentrimonium methosulfate.

4. A clear scalp composition according to claim 1 wherein the discrete particle size is from about 500 μm to about 7000 μm.

5. A clear scalp composition according to claim 1 wherein the discrete particle size is from about 1000 μm to about 5000 μm.

6. A clear scalp composition according to claim 1 wherein the scalp active is selected from the group consisting of piroctone olamine, climbazole, salicylic acid, zinc pyrithione, selenium sulfide, sulfur, menthol and mixtures thereof.

7. A clear scalp composition according to claim 6 wherein the scalp active is piroctone olamine.

8. A clear scalp composition according to claim 1 wherein the discrete particle comprises from about 0.25% to about 1% of piroctone olamine.

9. A clear scalp composition according to claim 1 wherein the pH is from about 3.5 to about 5.

10. A clear scalp composition according to claim 1 wherein the water soluble polymer is a cationic polymer.

11. A clear scalp composition according to claim 10 wherein the cationic polymer is polyquaternium-37.

12. A clear scalp composition according to claim 10 wherein the cationic polymer is a cationic guar polymer.

13. A clear scalp composition according to claim 10 wherein the cationic polymer is a polquaternium-10.

14. A clear scalp composition according to claim 10 wherein the cationic polymer is a diallyldimethylammonium chloride.

15. A clear scalp composition according to claim 1 wherein the water soluble polymer is present from about 0.05% to about 5%.

16. A clear scalp composition according to claim 15 wherein the water soluble polymer is present from about 0.3% to about 1%.

17. A clear scalp composition according to claim 1 wherein the aqueous carrier is selected from the group consisting of water, or a miscible mixture of water and an organic solvent.

18. A clear scalp composition according to claim 1 wherein the aqueous carrier is a surfactant selected from the group consisting of anionic, amphoteric, zwitterionic or non-ionic surfactants.

19. A clear scalp composition according to claim 1 wherein a swelling of the discrete particle is completed in sequence, by first soaking of the discrete particle separately in an aqueous solution for 0.5-3 hrs. and then adding to the clear scalp composition.

20. A clear scalp composition according to claim 1 wherein a swelling of the discrete particle is completed by simultaneously adding the discrete particle into the clear scalp composition to completely swell in 3-4 days.

21. A clear scalp composition according to claim 1 wherein the clear scalp composition is clear demonstrating demonstrates <50% haze.

22. A clear scalp composition according to claim 1 wherein the discrete particles are spreadable between fingers on a hand when sheared and is stable.

23. A clear scalp composition according to claim 1 wherein the discrete particle is a swellable particle which enables packing of the clear scalp composition on packing equipment.

24. A clear scalp composition according to claim 1 wherein the discrete particle swells from about 1.5 times to about 4 times its original size when combined with the clear scalp composition.

25. A clear scalp composition according to claim 1 wherein the discrete particle swells to 4 times its original size when combined with the clear scalp composition.
